(19) United States
(12) Patent Application Publication
(10) Pub. No.: US 2020/0368134 A1
(43) Pub. Date: Nov. 26, 2020

(54) POWDER COSMETIC COMPOSITION CONTAINING STARCH

(71) Applicant: COSMAX, INC., Hwaseong-si, Gyeonggi-do (KR)

(72) Inventors: Mi JANG, Seongnam-si, Gyeonggi-do (KR); Ki Sun KIM, Dongjak-gu, Seoul (KR); Kweon Jong YOO, Suwon-si, Gyeonggi-do (KR)

(73) Assignee: COSMAX, INC., Hwaseong-si, Gyeonggi-do (KR)

(21) Appl. No.: 16/758,951

(22) PCT Filed: Oct. 24, 2018
(86) PCT No.: PCT/KR2018/012626

§ 371 Date: Apr. 24, 2020

(30) Foreign Application Priority Data
Oct. 27, 2017 (KR) 10-2017-0141316

(51) Int. Cl. A61K 8/73; A61K 8/02; A61K 8/25; A61K 8/29; A61K 8/34; A61Q 1/10; A61Q 1/12; A61Q 1/06

(52) U.S. Cl. CPC A61K 8/732; A61K 8/022; A61K 8/25; A61K 8/29; A61K 2800/805; A61Q 1/10; A61Q 1/12; A61Q 1/06; A61K 8/345

(57) ABSTRACT

The present disclosure relates to a powder-to-cream type powder cosmetic composition. The powder cosmetic composition according to one aspect contains starch instead of hydrophobic silica, and thus not only has excellent lasting, moisturizing ability, and adhesiveness but also has an effect of being safe due to the harmlessness to the human body.

### TECHNICAL FIELD

The present disclosure relates to a powder-to-cream type powder cosmetic composition.

### BACKGROUND ART

In the case of a powder-to-cream type powder cosmetic composition, an aqueous phase component is captured inside powder having hydrophobicity, thus maintaining a powder phase. When such a cosmetic composition in the powder phase is applied to the skin, the aqueous phase component of the inside is mixed with the powder of the outside and thereby changed into a cream phase. Thus, the cosmetic composition may provide the effects of giving moisture to skin and increasing adhesion.

A capsulation method using hydrophobic silica, which is a technology of the related art, has disadvantages in that a user may feel dryness and powder flakes when used. In addition, in the case of a large number of hydrophobic silica, when used as nano-sized face powder, the hydrophobic silica has safety issues due to the possibility of inhalation thereof depending on the kind of applicator.

### DESCRIPTION OF EMBODIMENTS

### Technical Problem

An aspect provides the powder cosmetic composition containing starch, aqueous phase component, and powder which has excellent lasting, moisturizing, and adhering ability and is able to be safely used to a human body.

Another aspect provides a method of preparing the powder cosmetic composition including agitating powder; and spraying a mixture of starch and an aqueous phase components to the agitated powder, and then agitating the same.

### Solution to Problem

One aspect provides the powder cosmetic composition containing starch, an aqueous phase components, and powder.

The powder cosmetic composition may be a powder-to-cream type. The “powder-to-cream type” may mean a form which changes into a cream phase when a powder phase is applied to the skin. According to an embodiment, the powder cosmetic composition may have a powder phase as the viscosity of the aqueous components is increased by the starch and thereby the aqueous phase components are agglomerated and then powder is adsorbed to the outside of the agglomerated aqueous phase components. When the aqueous phase components having the viscosity increased by the starch are finely sprayed, the aqueous phase components are not rejoined to each other and agglomeration may be maintained, and hydrophobic powder may adhere to the outsides of the agglomerated aqueous phase components. Accordingly, when the powder cosmetic composition is applied to the skin, the aqueous phase components of the inside are mixed with the powder of the outside, and thereby the powder cosmetic composition may be changed into a cream phase. A powder-to-cream type powder cosmetic composition of a related art captures aqueous phase components inside hydrophobic silica and thus has disadvantages that a user feels dryness and powder flakes due to the hydrophobic silica. In addition, when the hydrophobic silica is used as a nano-sized face powder, safety issues occur due to possibility of inhalation. On the contrary, the powder cosmetic composition according to an embodiment contains starch instead of the hydrophobic silica, and thus has excellent lasting, moisturizing, and adhering ability, and may be safely used to a human body. According to an embodiment, the powder cosmetic composition containing starch has very excellent lasting, moisturizing, and adhering ability compared to a powder cosmetic composition containing hydrophobic silica or common face powder-type powder cosmetic composition.

In one embodiment, the starch may be contained in 0.1 wt % to 10.0 wt %, 0.1 wt % to 9.0 wt %, 0.1 wt % to 8.0 wt %, 0.1 wt % to 7.0 wt %, 0.1 wt % to 6.0 wt %, 0.1 wt % to 5.0 wt %, 1.0 wt % to 10.0 wt %, 1.0 wt % to 9.0 wt %, 1.0 wt % to 8.0 wt %, 1.0 wt % to 7.0 wt %, 1.0 wt % to 6.0 wt % or 1.0 wt % to 5.0 wt % with respect to the total weight of the composition. When starch content is less than 0.1 wt %, formation of formulation may be difficult because the effect on the aqueous phase components is insignificant. When the starch content is more than 10.0 wt %, the viscosity of the aqueous phase increases to the extent that dispersion is impossible and spraying is impossible, making it difficult to form a stable formulation and a problem in that a feeling of moisture decreases may occur.

The kind of the starch is not limited if the starch is uniformly dispersed and affects an increase of the viscosity of the aqueous phase when the starch is dispersed in an aqueous phase at room temperature. However, in particular, one or more kinds of the starch selected from a group consisting of rice starch, corn starch, potato starch, bean starch, and wheat starch may be used. For example, rice starch may be used.

The “rice starch” may mean starch obtained from seeds of rice (Oryza Sativa) as a raw material. The rice starch may be obtained from rice by the known method or commercially available rice starch can be purchased and used.

In one embodiment, the aqueous phase components may be contained in 1 wt % to 85 wt %, 5 wt % to 85 wt %, 10 wt % to 85 wt %, 20 wt % to 85 wt %, 30 wt % to 85 wt %, 40 wt % to 85 wt %, or 50 wt % to 85 wt % with respect to the total weight of the composition.

The aqueous phase components may contain moisturizing components. In one embodiment, the moisturizing components may be contained in 10 wt % to 50 wt % with respect to the total weight of the composition, but is not limited thereto.

The aqueous phase components may be a mixture of one or two or more selected from the group consisting of glycerin, diglycerin, propylene glycol, butylene glycol, polyethylene glycol, sorbitol, trehalose, raffinose, hyaluronic acid, and water, but is not limited thereto.

In one embodiment, the powder may be contained in 0.1 wt % to 20 wt %, 0.1 wt % to 15 wt %, 1 wt % to 20 wt %, 1 wt % to 15 wt %, 5 wt % to 20 wt %, 5 wt % to 15 wt %, 10 wt % to 20 wt %, or 10 wt % to 15 wt % of with respect to the total weight of the composition, but is not limited thereto.

The powder may be hydrophobic powder. A term, “hydrophobic”, means a property that lacks affinity for water. Accordingly, the hydrophobic powder may be a powder having hydrophobic groups.

The powder may contain extender pigments commonly used for a powder cosmetic composition. In one embodiment, the powder may be a mixture of one or two or more selected from the group consisting of talc, white mica, synthetic mica, sericite mica, kaolin, titanium dioxide, titanium oxide coated mica, aluminum powder, iron oxide, barium sulfate, methyl methacrylate crosspolymer, silica, vinyldimethicone/methicone silsesquioxane crosspolymer, polymethylsilsesquioxanes, mica/titanium dioxide, borosilicate/titanium dioxide, and synthetic fluorofluorophyte/titanium dioxide, but is not limited thereto.

The powder cosmetic composition may contain pigments commonly used for a powder cosmetic composition. In one embodiment, the pigments may be used as one or more components selected from iron oxide (red, yellow, black) titanium oxide, zinc oxide, red (No. 201 to No. 208, No. 213 to No. 215, No. 218 to No. 223, No. 225 to No. 228), yellow (No. 4, No. 5, No. 201 to No. 205), blue (No. 1, No. 2, No. 201 to No. 205, No. 403, No. 404), ultramarine, chromium oxide green, chromium hydroxide green, and manganese violet. In another embodiment, the pigments may be used as pigments surface-modified with silicone-based oil or the like.

The powder cosmetic composition may have a formulation selected from the group consisting of a two-way cake, a face powder, a compact, a makeup base, a skin cover, a concealer, an eye shadow, a cheek color, a lipstick, a lip gloss, a lip fixer, a mascara, and an eyebrow, but is not limited thereto.

The cosmetic composition according to an embodiment may also contain additional ingredients commonly used for cosmetics, for example, any common cosmetic components selected from thickeners, dispersants, fragrances, fillers, preservatives, antiseptics, neutralizing agents, sunscreens, sweeteners, vitamins, free-radical scavengers, metal ion sequestering agents, and a mixture thereof.

Those skilled in the art may select any additional ingredients and/or amounts thereof so that the advantageous properties of the composition according to the present disclosure are not adversely affected or substantially not adversely affected by the expected addition.

Another aspect provides a method for preparing the powder cosmetic composition including: agitating powder; and spraying a mixture of starch and aqueous phase components to the agitated powder, and then agitating the same.

The powder, the starch, the aqueous phase components, and the powder cosmetic composition are the same as stated above.

The method for preparing the powder cosmetic composition includes agitating powder. In agitating powder, agitation may be performed at 1,000 rpm to 3,000 rpm for 1 minute to 30 minutes, for example, at 1,500 rpm to 2,000 rpm for 1 minute to 10 minutes, but is not limited thereto.

The method for preparing the cosmetic powder composition includes spraying the mixture of the starch and the aqueous phase components to the agitated powder and then agitating the same. The spraying may be fine spraying. The spraying may be performed while agitation is performed at a speed of 500 rpm or lower. The agitation may be performed at 300 rpm to 700 rpm for 10 minutes or more, for example, 400 rpm to 600 rpm for 10 minutes or more, but is not limited thereto.

### Advantageous Effects of Disclosure

The powder cosmetic composition according to one aspect contains starch instead of hydrophobic silica and, thus, not only has excellent lasting, moisturizing, and adhering ability but also has an effect of being safe due to the harmlessness to a human body.

### MODE OF DISCLOSURE

Hereinafter, the present disclosure is described in more detail with reference to Examples. However, these Examples are for illustrative purposes only and the scope of the present disclosure is not limited to these Examples.

### Example 1: Preparation of the Powder Cosmetic Composition Containing Starch

A powder-to-cream type powder cosmetic composition containing rice powder is prepared. Table 1 shows the specific ingredients and content of the powder cosmetic composition prepared according to Example 1. Unless stated otherwise in the present specification, the component content is wt %.

TABLE 1

| | | Content |
|---|---|---|
| | Ingredients | (wt %) |
| 1 | Talc | 10.0 |
| 2 | Titanium dioxide | 5.0 |
| 3 | Silica | 2.5 |
| 4 | Vinyl dimethicone/methicone | 2.0 |
| | silsesquioxane crosspolymer | |
| 5 | Methyl methacrylate crosspolymer | 0.5 |
| 6 | Pigments | 1.5 |
| 7 | Rice starch | 3.0 |
| 8 | Purified water | To 100 |
| 9 | Glycerin | 15.0 |
| 10 | Butylene glycol | 10.0 |
| 11 | Antiseptic | Suitable |
| | | amount |

<Preparation Method>

1) The ingredients 1 to 6 are put in a Henschel mixer and mixed at 1700 rpm for 4 minutes.

2) The ingredients 7 and 8 are mixed by using an Agi mixer, and then the ingredients 9 to 11 are added thereto and mixed.

3) The mixture of 2) is finely sprayed to the mixture of 1). At this point, the mixtures are mixed by the Henschel mixer at the speed of 500 rpm or lower.

4) All of the mixture of 2) is sprayed and then dispersed at 500 rpm for 10 minutes or more.

### Comparative Example 1: Preparation of a Powder Cosmetic Composition Containing Hydrophobic Silica

A powder-to-cream type powder cosmetic composition containing hydrophobic silica is prepared. Table 2 shows the specific ingredients and content of the powder cosmetic composition prepared according to Comparative Example 1.

TABLE 2

| | Ingredients | Content |
|---|---|---|
| 1 | Silica silylate | 8.0 |
| 2 | Titanium dioxide | 5.0 |
| 3 | Silica | 2.5 |
| 4 | Vinyl Dimethicone/methicone | 2.0 |
| | silsesquioxane crosspolymer | |
| 5 | Methyl methacrylate crosspolymer | 0.5 |
| 6 | Pigments | 1.5 |
| 7 | Purified water | To 100 |
| 8 | Glycerin | 15.0 |
| 9 | Butylene glycol | 10.0 |
| 10 | Antiseptic | Suitable |
| | | amount |

<Preparation Method>

1) 1) The ingredients 1 to 6 are put in a Henschel mixer and mixed at 1700 rpm for 4 minutes.

2) The ingredients 7 to 10 are mixed by using an Agi mixer.

3) The mixture of 2) is finely sprayed to the mixture of 1). At this point, the mixtures are mixed by the Henschel mixer at the speed of 500 rpm or lower.

4) All of the mixture of 2) is sprayed and then dispersed at 500 rpm for 10 minutes or more.

### Comparative Example 2: Preparation of a Powder Cosmetic Composition

A common face powder-type powder cosmetic composition is prepared. Table 3 shows the specific ingredients and content of the powder cosmetic composition prepared according to Comparative Example 2.

TABLE 3

| | | Content |
|---|---|---|
| | Ingredients | (wt %) |
| 1 | Mica | To 100 |
| 2 | Mica/triethoxycaprylylsilane | 7.0 |
| 3 | Silica | 5.0 |
| 4 | Titanium dioxide | 2.0 |
| 5 | Methyl methacrylate crosspolymer | 4.3 |
| 6 | Dimethicone/vinyl dimethicone crosspolymer | 2.0 |
| 7 | Barium sulfate | 3.0 |
| 8 | Pigments | 1.5 |
| 9 | Dimethicone | 2.5 |
| 10 | Antiseptic | Suitable |
| | | amount |

<Preparation Method>

1) The ingredients 1 to 8 are put in a Henschel mixer and mixed at 1700 rpm for 4 minutes.

2) The ingredients 9 to 10 are heated and then finely sprayed to the mixture of 1).

### Experimental Example 1: Sensory Evaluation

A sensory evaluation test was performed on the powder cosmetic composition prepared according to Example 1 and Comparative Examples 1 and 2.

Specifically, a survey was conducted after each of 20 women in 20 to 40s were instructed to use, for 7 days, the powder cosmetic composition products prepared according to Example 1 and Comparative Examples 1 and 2. Application at least twice or more a day was recommended, and the content of the questionnaire consisted of questions for measuring lasting, moisturizing, and adhering ability. All the evaluations were rated from 0 to 10 points. In the sensory evaluation, an average score was marked by scoring 10 points for the positive parts (excellent lasting ability, no dryness, and excellent adhering ability) and 0 points for the negative parts (low lasting ability, dryness, low adhering ability). Table 4 shows the sensory evaluation results of the powder cosmetic composition prepared according to Example 1 and Comparative Examples 1 to 2.

TABLE 4

| | | Comparative | Comparative |
|---|---|---|---|
| Items | Example | Example 1 | Example 2 |
| Lasting ability | 8.5 | 7.0 | 5.0 |
| Moisturizing sensation | 8.7 | 7.2 | 4.2 |
| Adhering ability | 8.3 | 7.5 | 5.2 |

As shown in Table 4, the overall satisfaction with the lasting ability, moisturizing sensation, and adhering ability of the powder cosmetic composition according to Example 1 was higher than the powder cosmetic composition according to Comparative Examples 1 and 2. In particular, Comparative Example 2, which is a common face powder, was identified to have very poor lasting ability and moisturizing sensation.

1. The powder cosmetic composition containing starch, an aqueous phase component, and powder.

2. The powder cosmetic composition according to claim 1, wherein the powder cosmetic composition has a powder phase as the viscosity of the aqueous component is increased by the starch and thereby the aqueous phase components are agglomerated and then powder is adsorbed to the outside of the agglomerated aqueous phase component.

3. The powder cosmetic composition according to claim 1, wherein the starch is contained in 0.1 wt % to 10.0 wt % with respect to the total weight of the composition.

4. The powder cosmetic composition according to claim 1, wherein the starch is one or more kinds selected from the group consisting of rice starch, corn starch, potato starch, bean starch, and wheat starch.

5. The powder cosmetic composition according to claim 1, wherein the aqueous phase component is contained in 1 wt % to 85% with respect to the total weight of the composition.

6. The powder cosmetic composition according to claim 1, wherein the aqueous phase component is a mixture of one or two or more selected from the group consisting of glycerin, diglycerin, propylene glycol, butylene glycol, polyethylene glycol, sorbitol, trehalose, raffinose, hyaluronic acid, and water.

7. The powder cosmetic composition according to claim 1, wherein the powder is contained in 0.1 wt % to 20 wt % with respect to the total weight of the composition.

8. The powder cosmetic composition according to claim 1, wherein the powder is a mixture of one or two or more selected from the group consisting of talc, white mica, synthetic mica, sericite mica, kaolin, titanium dioxide, titanium oxide-coated mica, aluminum powder, iron oxide, barium sulfate, methyl methacrylate crosspolymer, silica, vinyldimethicone/methicone silsesquioxane crosspolymer, polymethylsilsesquioxanes, mica/titanium dioxide, borosilicate/titanium dioxide, and synthetic fluorofluorophyte/titanium dioxide.

9. The powder cosmetic composition according to claim 1, wherein the powder cosmetic composition has a formulation selected from the group consisting of a two-way cake, a face powder, a compact, a makeup base, a skin cover, a concealer, an eye shadow, a cheek color, a lipstick, a lip gloss, a lip fixer, a mascara, and an eyebrow.

10. A method for preparing the powder cosmetic composition according to claim 1, the method comprising:
agitating powder; and
spraying a mixture of starch and an aqueous phase component to the agitated powder, and then agitating the same.
